(19) United States
(12) Patent Application Publication
(10) Pub. No.: US 2008/0262191 A1
(43) Pub. Date: Oct. 23, 2008

(54) METHODS FOR THE PREPARATION OF IMIDES, MALEIMIDES AND MALEIMIDE-TERMINATED POLYIMIDE COMPOUNDS

(76) Inventor: Farhad G. Mizori, La Mesa, CA (US)

Correspondence Address: The Law Office of Jane K. BABIN,;Professional Corporation, c/o Intellevate, P.O. Box 52050, Minneapolis, MN, 55402, US

(21) Appl. No.: 12/021,283

(22) Filed: Jan. 28, 2008

Related U.S. Application Data
(60) Provisional application No. 60/897,693, filed on Jan. 26, 2007.
(60) Provisional application No. 60/897,692, filed on Jan. 26, 2007.
(60) Provisional application No. 60/922,422, filed on Apr. 9, 2007.

(51) Int. Cl. C08G 73/10; C07D 207/448

(52) U.S. Cl. 528/329.1; 548/548; 548/549

(57) ABSTRACT

Provided herein are methods for preparing maleimides, polyimides and maleimide-terminated polyimides. Specifically disclosed are methods for preparing maleimides from N-substituted maleamic acids in the presence of an alcohol catalyst and an acid co-catalyst in a solvent. Also disclosed are methods for preparing polyimides by reacting a diamine with a dianhydride in a reaction medium that includes a solvent and a polar protic co-solvent. Further provided are methods for preparing maleimide-terminated polyimides from diamines in an aromatic solvent containing a polar protic co-solvent.

### RELATED APPLICATIONS

The present application claims the benefit of priority under 35 U.S.C. § 119 to U.S. Provisional Application Nos. 60/897,692, filed Jan. 26, 2007; 60/897,693, filed Jan. 26, 2007; and 60/922,422, filed Apr. 9, 2007, the contents of which are incorporated herein by reference in their entirety.

### BACKGROUND OF THE INVENTION

Polyimides are a class of polymers that contain imide repeat units. As thermoplastic polymers, polyimides are noted for their strength, temperature resistance and chemical resistance. Maleimides are a subset of imides that additionally contain a polymerizable olefinic functionality. Maleimides are an important class of thermoset imide containing resins used extensively in the fields of electronic packaging, aerospace adhesive and matrix resins, and automotive applications.

### Preparation of Maleimides

Several methods have been described for the preparation of maleimides. In general, preparation of a maleimide starts with an initial reaction of an amine with maleic anhydride to form a maleamic acid. Ring-closure of the maleamic acid then provides the desired maleimide, as shown below. The ring-closure is the most difficult step in the synthesis of maleimides and is especially difficult to achieve where the parent amine is directly attached to an aliphatic backbone.

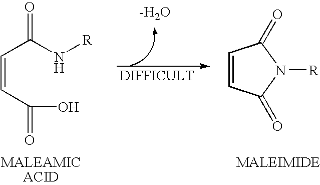

Few methods have been reported to accomplish the ring-closure step and none has proven wholly satisfactory. One method is through the reaction of the maleamic acid residues with acetic anhydride and sodium acetate. This reaction tends to work well with aromatic maleimides that can be purified by recrystallization. However, this synthetic method has proven troublesome when trying to prepare aliphatic maleimides, resulting in low yield and purity of the desired end product. Furthermore, crude maleimide reaction products made using aliphatic amine starting materials, via acetic anhydride plus sodium acetate, are usually dark purple or black in color, which may be undesirable in certain circumstances.

Another method often used is the reaction of the maleamic acid with another dehydrating agent such as dicyclohexylcarbodiimide (DCC). DCC closes maleamic acids much more readily than does acetic anhydride. With DCC alone, the product is exclusively an isomaleimide. However, in the presence of a suitable isomerizing agent, such as 1-hydroxybenzotriazole (HOBt), the maleimide is produced instead of the isomaleimide. It has been postulated that the HOBt functions in the reaction to allow ring-closure to proceed via an HOBt ester of the maleamic acid (formed via the agency of DCC), which presumably closes preferentially to the maleimide. Likely, isomerizing agents such as HOBt add to the isoimide to yield the amic acid ester. If the amic acid ester exhibits any tendency to close to the imide, much less a strong bias for doing so, a route for interconverting isoimide and imide would thereby be established, with the thermodynamically favored imide product ultimately prevailing. Thus, if the initial closure of ester formed in the DCC reaction yields isoimide, or if any isoimide is produced by direct closure of the acid, the situation would be subsequently “corrected” via conversion of the isoimide to the imide by the action of the active ester alcohol as an isomerizing agent. Although this is a mild reaction pathway to maleimide production, DCC poses a health risks as a potent allergen and sensitizer. Furthermore, the side product, dicyclohexylurea (DCU), is difficult to separate from the maleimide.

Another method that has been employed for ring-closure involves thermal cyclodehydration of the maleamic acid using an acid catalyst combined with an azeotroping solvent. The reaction rate is slow, and a polar aprotic solvent is typically used along with the acid catalyst in order to conclude the reaction in a reasonable amount of time often. A difficulty inherent with this reaction pathway is the removal of the polar aprotic solvent. Polar aprotic solvents typically have high boiling points, are relatively expensive, and can pose serious health and environmental risks.

### Preparation of Polyimides

The classic synthesis of polyimides starts with the reaction of one or more diamines with one or more dianhydrides in a polar aprotic solvent or combination of solvents, such as toluene or xylenes plus a polar aprotic solvent. The intermediate that is formed is a polyamic acid, which has very poor solubility in toluene or xylenes. Polar aprotic solvents are included in the solution to improve solubility of the polyamic acid.

Commercial polyimide resins are often prepared by casting polyamic acid solutions in thin films. Application of heat promotes a cyclodehydration reaction resulting in the desired polyimide. Although the imide ring is far easier to form than a maleimide ring, the ring cyclodehydration procedure using heat is often accompanied by incomplete ring closure. As a result of incomplete ring closure, polyimides cast and cyclodehydrated in this fashion are reported to absorb large amounts of moisture.

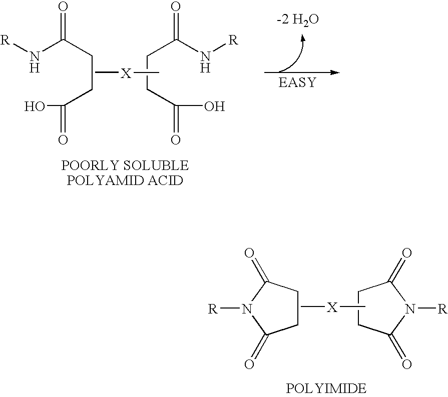

In addition, polar aprotic solvents typically used in the preparation of polyimides (such as N-methylpyrrolidone (NMP), dimethylformamide (DMF), and DMAC), have very high boiling and are therefore difficult to remove from the polymer. These solvents are also readily absorbed through the skin and possess health hazards.

Thus, there remains a need for inexpensive, efficient, safe and environmentally friendly methods to synthesize useful maleimide and polyimide compounds in highly pure form.

### SUMMARY OF THE INVENTION

The present invention provides methods for preparing maleimides, polyimides and maleimide-terminated polyimides. In particular, the present invention provides a methods for preparing a maleimide, including the steps of contacting an N-substituted maleamic acid with an alcohol catalyst and an acid co-catalyst in a solvent, where the alcohol catalyst has a boiling point equal to or greater than the boiling point of the solvent, and heating the reaction medium at an elevated temperature, resulting in cyclodehydration of the N-substituted maleamic acid, and thereby preparing a maleimide.

The alcohol catalyst can be a C₄ to about C₁₀ alcohol and, in certain embodiments is present in the reaction medium in the range of 10 mol % to about 40 mol %, based on mol % of N-substituted maleamic acid. In certain embodiments, the alcohol catalyst is a C₄ alcohol, such as n-butanol. In other embodiments, the alcohol catalyst is a C₅ alcohol, such as n-pentanol.

According to this method of the invention, the acid co-catalyst can be an organic sulfonic acid, such as benzenesulfonic acid, para-toluenesulfonic acid, para-bromobenzenesulfonic acid, para-nitrobenzenesulfonic acid, ethylbenzenesulfonic acid, methanesulfonic acid, ethanesulfonic acid, propanesulfonic acid, trifluoromethanesulfonic acid, nonafluorobutanesulfonic acid, or 2,2,2-trifluoroethanesulfonic acid.

In certain aspects of this method, the solvent has a boiling point of at least about 100° C. In yet further aspects of the invention, the solvent can be an alkane, a cycloalkane, an aromatic hydrocarbon or halogen-substituted derivative thereof. For example, the solvent can be toluene.

The present invention also provides a method for preparing a maleimide-terminated polyimide, including the steps of contacting at least one diamine with at least one anhydride in an aromatic solvent containing at least one polar protic co-solvent, wherein a polyamic acid is formed, then contacting the polyamic acid with maleic anhydride, to form maleamic acid termini on the intermediate, followed by acid catalysis to close the maleamic acid rings to form a polyimide.

In certain embodiments of this method of the invention, the acid catalyst comprises a polymer-supported acid catalyst which can be conveniently removed by gravity filtration at the end of the synthesis.

The at least one diamine can include, for example, 1,10-diaminodecane; 1,12-diaminododecane; dimer diamine; 1,2-diamino-2-methylpropane; 1,2-diaminocyclohexane; 1,2-diaminopropane; 1,3-diaminopropane; 1,4-diaminobutane; 1,5-diaminopentane; 1,7-diaminoheptane; 1,8-diaminomenthane; 1,8-diaminooctane; 1,9-diaminononane; 3,3′-diamino-N-methyldipropylamine; diaminomaleonitrile; 1,3-diaminopentane; 9,10-diaminophenanthrene; 4,4′-diaminooctafluorobiphenyl; 3,5-diaminobenzoic acid; 3,7-diamino-2-methoxyfluorene; 4,4′-diaminobenzophenone; 3,4-diaminobenzophenone; 3,4-diaminotoluene; 2,6-diaminoanthroquinone; 2,6-diaminotoluene; 2,3-diaminotoluene; 1,8-diaminonaphthalene; 2,4-diaminotoluene; 2,5-diaminotoluene; 1,4-diaminoanthroquinone; 1,5-diaminoanthroquinone; 1,5-diaminonaphthalene; 1,2-diaminoanthroquinone; 2,4-cumenediamine; 1,3-bisaminomethylbenzene; 1,3-bisaminomethylcyclohexane; 2-chloro-1,4-diaminobenzene; 1,4-diamino-2,5-dichlorobenzne; 1,4-diamino-2,5-dimethylbenzene; 4,4′-diamino-2,2′-bistrifluoromethylbiphenyl; bis(amino-3-chlorophenyl)ethane; bis(4-amino-3,5-dimethylphenyl)methane; bis(4-amino-3,5-diethylphenyl)methane; bis(4-amino-3-ethyl diaminofluorene; diaminobenzoic acid; 2,3-diaminonaphthalene; 2,3-diaminophenol; -5-methylphenyl)methane; bis(4-amino-3-methylphenyl)methane; bis(4-amino-3-ethylphenyl)methane; 4,4′-diaminophenylsulfone; 3,3′-diaminophenylsulfone; 2,2-bis(4, -(4-aminophenoxy)phenyl)sulfone; 2,2-bis(4-(3-aminophenoxy)phenyl)sulfone; 4,4′-oxydianiline; 4,4′-diaminodiphenyl sulfide; 3,4′-oxydianiline; 2,2-bis(4-(4-aminophenoxy)phenyl)propane; 1,3-bis(4-aminophenoxy)benzene; 4,4′-bis(4-aminophenoxy)biphenyl; 4,4′-diamino-3,3′-dihydroxybiphenyl; 4,4′-diamino-3,3′-dimethylbiphenyl; 4,4′-diamino-3,3′-dimethoxybiphenyl; Bisaniline M; Bisaniline P; 9,9-bis(4-aminophenyl)fluorene; o-tolidine sulfone; methylene bis(anthranilic acid); 1,3-bis(4-aminophenoxy)-2,2-dimethylpropane; 1,3-bis(4-aminophenoxy)propane; 1,4-bis(4-aminophenoxy)butane; 1,5-bis(4-aminophenoxy)butane; 2,3,5,6-tetramethyl-1,4-phenylenediamine; 3,3′,5,5′-tetramethylbenzidine; 4,4′-diaminobenzanilide; 2,2-bis(4-aminophenyl)hexafluoropropane; polyoxyalkylenediamines (e.g. Huntsman's Jeffamine D-230, D400, D-2000, and D-4000 products); 1,3-cyclohexanebis(methylamine); m-xylylenediamine; p-xylylenediamine; bis(4-amino-3-methylcyclohexyl)methane; 1,2-bis(2-aminoethoxy)ethane; 3(4), 8(9)-bis(aminomethyl)tricyclo(5.2.1.0^{2,6})decane; or isophoronediamine.

The at least one anhydride for preparation of maleimide-terminated polyimides can include, for example, polybutadiene-graft-maleic anhydride; polyethylene-graft-maleic anhydride; polyethylene-alt-maleic anhydride; polymaleic anhydride-alt-1-octadecene; polypropylene-graft-maleic anhydride; poly(styrene-co-maleic anhydride); pyromellitic dianhydride; maleic anhydride, succinic anhydride; 1,2,3,4-cyclobutanetetracarboxylic dianhydride; 1,4,5,8-naphthalenetetracarboxylic dianhydride; 3,4,9,10-perylenetetracarboxylic dianhydride; bicyclo(2.2.2)oct-7-ene-2,3,5,6-tetracarboxylic dianhydride; diethylenetriaminepentaacetic dianhydride; ethylenediaminetetraacetic dianhydride; 3,3′, 4,4′-benzophenone tetracarboxylic dianhydride; 3,3′,4,4′-biphenyl tetracarboxylic dianhydride; 4,4′-oxydiphthalix anhydride; 3,3′,4,4′-diphenylsulfone tetracarboxylic dianhydride; 2,2′-bis(3,4-dicarboxyphenyl)hexafluoropropane dianhydride; 4,4′-bisphenol A diphthalic anhydride; 5-(2,5-dioxytetrahydro)-3-methyl-3-cyclohexene-1,2-dicarboxylic anhydride; ethylene glycol bis(trimellitic anhydride); hydroquinone diphthalic anhydride; allyl nadic anhydride; 2-octen-1-ylsuccinic anhydride; phthalic anhydride; 1,2,3,6-tetrahydrophthalic anhydride; 3,4,5,6-tetrahydrophthalic anhydride; 1,8-naphthalic anhydride; glutaric anhydride; dodecenylsuccinic anhydride; hexadecenylsuccinic anhydride; hexahydrophthalic anhydride; methylhexahydrophthalic anhydride; or tetradecenylsuccinic anhydride. In certain embodiments of the invention, the aromatic solvent is toluene and/or the at least one polar protic co-solvent includes formic acid.

### DETAILED DESCRIPTION

It is to be understood that both the foregoing general description and the following detailed description are exemplary and explanatory only and are not restrictive of the invention claimed. As used herein, the use of the singular includes the plural unless specifically stated otherwise. As used herein, “or” means “and/or” unless stated otherwise. Furthermore, use of the term “including” as well as other forms, such as “includes,” and “included,” is not limiting.

The section headings used herein are for organizational purposes only and are not to be construed as limiting the subject matter described.

Unless defined otherwise, all technical and scientific terms used herein have the same meaning as is commonly understood by one of skill in the art to which the claimed subject matter belongs. In the event that there are a plurality of definitions for terms herein, those in this section prevail.

Unless specific definitions are provided, the nomenclatures utilized in connection with, and the laboratory procedures and techniques of analytical chemistry and synthetic organic chemistry described herein are those known in the art. Standard techniques can be used for chemical syntheses and analyses. The foregoing techniques and procedures can be generally performed according to conventional methods well known in the art.

Whenever it appears herein, a numerical range such as “1 to 20” refers to each integer in the given range; e.g., “1 to 20 carbon atoms” means that an alkyl group can contain only 1 carbon atom, 2 carbon atoms, 3 carbon atoms, etc., up to and including 20 carbon atoms, although the term “alkyl” also includes instances where no numerical range of carbon atoms is designated).

“Alkane,” as used herein, refers to saturated straight-chain, branched or cyclic hydrocarbons having only single bonds. Alkanes have general formula CₙH₂ₙ₊₂.

“Cycloalkane,” refers to an alkane having one or more rings in its structure.

“Aromatic hydrocarbon,” as used herein, refers to compounds having one or more benzene rings.

“Halogen,” as used herein, includes fluorine, chlorine, bromine and iodine.

“Imide” as used herein, refers to a functional group having two carbonyl groups bound to a primary amine or ammonia. The general formula of an imide of the invention is:

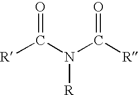

“Polyimides” are polymers of imide-containing monomers. Polyimides typically have one of two forms: linear or cyclic. Non-limiting examples of linear and cyclic (e.g. an aromatic heterocyclic polyimide) polyimides are shown below for illustrative purposes.

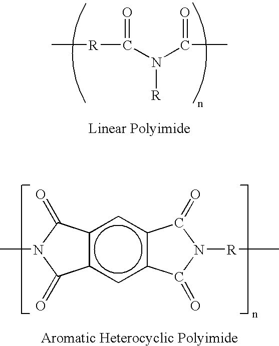

“Maleimide,” as used herein, refers to an N-substituted maleimide having the formula as shown below:

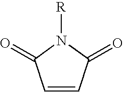

Wherein the “R” group may be an aromatic, heteroaromatic, aliphatic, or polymeric moiety.

“Diamine,” as used herein, refers generally to a compound or mixture of compounds, where each species has 2 amine groups.

The term “solvent,” as used herein, refers to a liquid that dissolves a solid, liquid, or gaseous solute, resulting in a solution. “Co-solvent” refers to a second, third, etc. solvent used with a primary solvent.

As used herein, “polar protic solvents” are ones that contains an O—H or N—H bond, while “polar aprotic solvents” do not contain an O—H or N—H bond.

As used herein, “alcohol catalyst” refers to an alcohol or combination of alcohols that, when added to a chemical reaction, has the effect of accelerating, increasing the rate or yield of the reaction without being consumed by the overall reaction. Typically, an alcohol catalyst will contain a single alcohol, but mixtures comprising two or more alcohols are contemplated for use in the present invention. As used herein, “acid catalyst” refers to any acidic substance or compound that, when added to a chemical reaction, has the effect of accelerating, increasing the rate or yield of the reaction without being consumed by the overall reaction. Typically, an acid catalyst will contain a single acid, but mixtures comprising two or more acids are contemplated for use in the present invention. Acid catalysts of the invention can be soluble or insoluble. For example, polymer-bound acid catalysts may conveniently be used in the methods of the invention and then easily removed e.g. by gravity filtration.

The present invention provides methods for the synthesis of imides, maleimides, and maleimide-terminated polyimides.

### Preparation of Maleimides

In one embodiment, the present invention provides methods for the preparation of maleimides based on the theory of converting maleamic acid residues to esters in the presence of an acid catalyst. Theoretically, the kinetics of the conversion under these conditions favor ring-closure of the maleamic acid to form a maleimide, with concomitant elimination of an alcohol. However, attempts at carrying out this scheme using low molecular weight alcohols, such as methanol and ethanol, have not resulted in an appreciable reaction.

The present invention is based on the discovery that higher boiling point alcohols can be used to efficiently prepare maleimide compounds. When an alcohol is used that has a boiling point equal to or higher than the solvent (e.g. toluene), the alcohol does not distill with water and is not removed from the reaction mixture, but instead serves as a catalyst. Thus, the present invention provides methods for converting maleamic acid residues to esters in the presence of an acid catalyst and an alcohol catalyst as shown below in Scheme I.

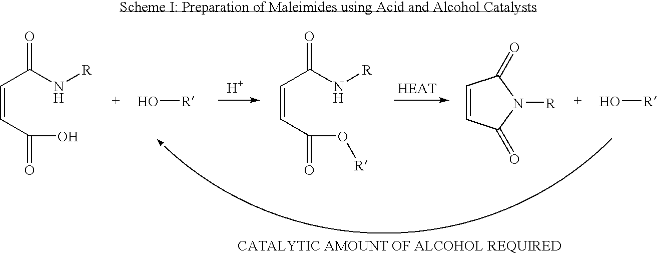

In certain embodiments of the invention, the alcohol catalyst has a boiling point that is about the same as the boiling point of the solvent used in the reaction. In other embodiments, the alcohol catalyst has a boiling point higher than the boiling point of the solvent used in the reaction.

Acid catalysts suitable for use in the practice of the invention include, but are not limited to, sulfonic acids, carboxylic acids, polymer supported acid catalysts, such as Amberlyst resins, and the like or mixtures thereof. In certain aspects of the invention, the use of any acid catalyst that is suitable for the synthesis of esters is contemplated for use in the methods of the invention.

Alcohols contemplated for use as catalysts, alone or in combinations, in the practice of the invention include, but are not limited to, linear alcohols, branched alcohols, cyclic alcohols, polyols, and the like. In certain aspects of the invention, one or more alcohol of the alcohol catalyst may contain additional oxygen in the backbone in the form of ether linkages. Alcohols containing other heteroatoms (e.g. N and/or S) are also suitable for use in the invention. Further contemplated are such alcohols as 2-ethoxyethanol, which boils at 135° C. and is completely miscible with water; 2-methoxyethanol (bp 124° C.); 2-(2-methoxyethoxy)ethanol (bp 193° C.); and 2-phenoxyethanol (247° C.).

In certain embodiments of the invention, the alcohol has a boiling point at least about 110° C., at least about 115° C., at least about 120° C., at least about 125° C., at least about 130° C., at least about 135° C., or at least about 140° C.

According to certain embodiments of the invention, the alcohol catalyst includes at least one C₄ to about C₁₀ alcohol. In certain aspects of the invention, the alcohol catalyst includes a C₄ alcohol such as n-butanol. In other aspects, the alcohol catalyst includes a C₅ alcohol such as n-pentanol.

In certain embodiments of the invention, the alcohol catalyst is added to the reaction mixture in a range of less than about 1 mol % up to large excess, for example greater than about 100 mol %, relative to the equivalents of amic or maleamic acid residues present. In other embodiments, the added alcohol catalyst comprises at least about 10 mol %, at least about 20 mol %, at least about 30 mol %, at least about 40 mol %, at least about 50 mol %, at least about 60 mol %, at least about 70 mol %, at least about 80 mol %, at least about 90 mol % or more, relative to the equivalents of amic or maleamic acid residues present. In one embodiment of the invention, the alcohol catalyst is present in the reaction medium in the range of 10 mol % to about 40 mol %, based on mol % of N-substituted maleamic acid.

### Preparation of Polyimides

The present invention also provides methods for the preparation of polyimides. These methods are based on the discovery that the addition of at least one polar protic solvents, such as acetic acid and formic acid, to toluene or xylenes improves the synthetic reactions for the preparation of polyimides. Formic acid and acetic acid boil at lower temperatures than the classic polar aprotic solvents. Thus, formic acid and acetic acid are much easier to remove from the reaction mixture. An additional advantage is that formic acid and acetic acid azeotrope with the water formed in the reaction to provide the desired polyimide in toluene or xylenes without contamination from an undesired solvent.

In one embodiment, the invention provides a method for preparing polyimides by reacting at least one diamine with at least one dianhydride in a reaction medium that includes a solvent and a polar protic co-solvent. In certain aspects of this method, the at least one polar protic co-solvent increases the solubility of the polyamic acid intermediate when compared to the solvent alone. Exemplary polar protic solvents include formic acid (bp 100-101° C.) and acetic acid (bp 117-118° C.). Both of these acids are inexpensive, readily available, infinitely soluble in water, and they have sufficiently low boiling points to be easily removed from a polymer mixture.

According to this method of the invention, a polyamic acid intermediate is formed from the diamine and a dianhydride. When the polyamic acid ring closes to form a polyimide according to this method of the invention, water that is eliminated from the reaction can be collected in a Dean-Stark trap. Advantageously, all of the polar protic co-solvent (e.g. formic or acetic acid) added to the reaction mixture can be collected along with the eliminated water. Thus, a highly pure solution of a polyimide containing minimal amounts of impurities can be prepared according to this method of the invention. The polyimide prepared according to certain methods of the invention can be recovered in an aromatic solvent such as, toluene or xylenes, which can be flashed off very easily resulting in a polyimide film that does not require high temperatures to process.

Typically, the reaction will contain a single diamine and a single dianhydride. However, the skilled artisan will recognize that combinations of these starting materials can produce polyimides having desirable properties. For example, dimer diamine imparts a certain amount of flexibility to a polyimide, while the tricyclodecane diamine provides high glass transition. The combination of these two diamines gives a polyimide backbone with superior toughness. Varying the amounts of the two diamine starting compounds in a polyimide reaction of the invention permits a high degree of control over the properties of the resulting polyimide

In certain embodiments, the solvent for synthesis of polyimide is toluene (bp 110° C.) and formic acid (bp 101° C.) is added as co-solvent. In other embodiments, xylenes (bp 135° C.) are used as the solvent with the addition of formic acid and/or acetic acid (bp 118° C.) as co-solvents. The skilled artisan will recognize that various solvents and combinations of solvents can be used in methods of the invention. Furthermore, mixtures of polar protic co-solvents are also contemplated.

The amount of polar protic solvent used in the reaction will depend on the characteristics of the polyamic acid intermediate that is formed. Typically, the polar protic solvent is used in the range of 5% up to about 50%, which can be, for example, about 5%, about 10%, about 15%, about 20%, about 25%, about 30%, about 35%, about 40%, about 45%, or about 50%.

### Preparation of Maleimide-Terminated Polyimides

The present invention also provides methods for synthesizing maleimide-terminated polyimides as highly pure solutions in toluene, xylene or other aromatic solvents. An exemplary maleimide-terminated polyimide synthesized according to the methods of the present invention will have the generic formula shown below:

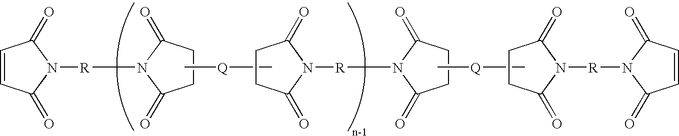

According to one embodiment of this method of the invention, a polyamic acid is formed in an aromatic solvent containing a polar protic co-solvent, such as formic acid or acetic acid. Polyimides are then formed by the addition of maleic anhydride to the amine end groups, followed by ring closure in the presence of an acid catalyst. Water and the polar protic co-solvent are then eliminated.

In certain aspects of this method, the acid catalyst is a polymer-supported acid catalyst such as Amberlyst-70 acidic ion exchange resin. When the catalyst is polymer-supported, the maleimide-terminated polyimide solution can be purified by simple gravity filtration to remove the catalyst from the solution.

Diamines suitable for use in the methods of the present invention include, but are not limited to 1,10-diaminodecane; 1,12-diaminododecane; dimer diamine; 1,2-diamino-2-methylpropane; 1,2-diaminocyclohexane; 1,2-diaminopropane; 1,3-diaminopropane; 1,4-diaminobutane; 1,5-diaminopentane; 1,7-diaminoheptane; 1,8-diaminomenthane; 1,8-diaminooctane; 1,9-diaminononane; 3,3′-diamino-N-methyldipropylamine; diaminomaleonitrile; 1,3-diaminopentane; 9,10-diaminophenanthrene; 4,4′-diaminooctafluorobiphenyl; 3,5-diaminobenzoic acid; 3,7-diamino-2-methoxyfluorene; 4,4′-diaminobenzophenone; 3,4-diaminobenzophenone; 3,4-diaminotoluene; 2,6-diaminoanthroquinone; 2,6-diaminotoluene; 2,3-diaminotoluene; 1,8-diaminonaphthalene; 2,4-diaminotoluene; 2,5-diaminotoluene; 1,4-diaminoanthroquinone; 1,5-diaminoanthroquinone; 1,5-diaminonaphthalene; 1,2-diaminoanthroquinone; 2,4-cumenediamine; 1,3-bisaminomethylbenzene; 1,3-bisaminomethylcyclohexane; 2-chloro-1,4-diaminobenzene; 1,4-diamino-2,5-dichlorobenzne; 1,4-diamino-2,5-dimethylbenzene; 4,4′-diamino-2,2′-bistrifluoromethylbiphenyl; bis(amino-3-chlorophenyl)ethane; bis(4-amino-3,5-dimethylphenyl)methane; bis(4-amino-3,5-diethylphenyl)methane; bis(4-amino-3-ethyl diaminofluorene; diaminobenzoic acid; 2,3-diaminonaphthalene; 2,3-diaminophenol; -5-methylphenyl)methane; bis(4-amino-3-methylphenyl)methane; bis(4-amino-3-ethylphenyl)methane; 4,4′-diaminophenylsulfone; 3,3′-diaminophenylsulfone; 2,2-bis(4, -(4-aminophenoxy)phenyl)sulfone; 2,2-bis(4-(3-aminophenoxy)phenyl)sulfone; 4,4′-oxydianiline; 4,4′-diaminodiphenyl sulfide; 3,4′-oxydianiline; 2,2-bis(4-(4-aminophenoxy)phenyl)propane; 1,3-bis(4-aminophenoxy)benzene; 4,4′-bis(4-aminophenoxy) biphenyl; 4,4′-diamino-3,3′-dihydroxybiphenyl; 4,4′-diamino-3,3′-dimethylbiphenyl; 4,4′-diamino-3,3′-dimethoxybiphenyl; Bisaniline M; Bisaniline P; 9,9-bis(4-aminophenyl)fluorene; o-tolidine sulfone; methylene bis(anthranilic acid); 1,3-bis(4-aminophenoxy)-2,2-dimethylpropane; 1,3-bis(4-aminophenoxy)propane; 1,4-bis(4-aminophenoxy)butane; 1,5-bis(4-aminophenoxy)butane; 2,3,5,6-tetramethyl-1,4-phenylenediamine; 3,3′,5,5′-tetramethylbenzidine; 4,4′-diaminobenzanilide; 2,2-bis(4-aminophenyl)hexafluoropropane; polyoxyalkylenediamines (e.g. Huntsman's Jeffamine D-230, D400, D-2000, and D-4000 products); 1,3-cyclohexanebis(methylamine); m-xylylenediamine; p-xylylenediamine; bis(4-amino-3-methylcyclohexyl)methane; 1,2-bis(2-aminoethoxy)ethane; 3(4), 8(9)-bis(aminomethyl)tricyclo(5.2.1.0^{2,6})decane; α,ω-bis(3-aminopropyl)polydimethylsiloxane; isophoronediamine and the like.

Anhydrides suitable for use in the methods of the invention, include but are not limited to: polybutadiene-graft-maleic anhydride; polyethylene-graft-maleic anhydride; polyethylene-alt-maleic anhydride; polymaleic anhydride-alt-1-octadecene; polypropylene-graft-maleic anhydride; poly(styrene-co-maleic anhydride); pyromellitic dianhydride; maleic anhydride, succinic anhydride; 1,2,3,4-cyclobutanetetracarboxylic dianhydride; 1,4,5,8-naphthalenetetracarboxylic dianhydride; 3,4,9,10-perylenentetracarboxylic dianhydride; bicyclo(2.2.2)oct-7-ene-2,3,5,6-tetracarboxylic dianhydride; diethylenetriaminepentaacetic dianhydride; ethylenediaminetetraacetic dianhydride; 3,3′,4,4′-benzophenone tetracarboxylic dianhydride; 3,3′,4,4′-biphenyl tetracarboxylic dianhydride; 4,4′-oxydiphthalix anhydride; 3,3′,4,4′-diphenylsulfone tetracarboxylic dianhydride; 2,2′-bis(3,4-dicarboxyphenyl)hexafluoropropane dianhydride; 4,4′-bisphenol A diphthalic anhydride; 5-(2,5-dioxytetrahydro)-3-methyl-3-cyclohexene-1,2-dicarboxylic anhydride; ethylene glycol bis(trimellitic anhydride); hydroquinone diphthalic anhydride; allyl nadic anhydride; 2-octen-1-ylsuccinic anhydride; phthalic anhydride; 1,2,3,6-tetrahydrophthalic anhydride; 3,4,5,6-tetrahydrophthalic anhydride; 1,8-naphthalic anhydride; glutaric anhydride; dodecenylsuccinic anhydride; hexadecenylsuccinic anhydride; hexahydrophthalic anhydride; methylhexahydrophthalic anhydride; tetradecenylsuccinic anhydride; and the like.

The invention will be further illustrated by reference to the following non-limiting Examples.

### EXAMPLES

### Example 1

### Preparation of Bismaleimide in the Presence of 25 mol % Alcohol Catalyst

To a 500-mL round bottomed flask equipped with a Teflon-coated magnetic stir bar, Dean-Stark trap and reflux condenser was added 20.1 g of maleic anhydride along with 300-mL of toluene. The mixture was stirred at room temperature until the maleic anhydride was fully dissolved. This was followed by the slow addition of 54.0 g of dimer diamine (Versamine-552, Cognis Corporation), while maintaining the solution at room temperature. To the amic acid solution was added 4.4 g of n-pentanol, followed by the addition of 10.0 g of methanesulfonic acid. The solution was heated and maintained at reflux for 24 hours, during which the predicted amount of water was collected in the Dean-Stark trap. The toluene was removed under vacuum and the product was extracted from the sludge with 300-mL of heptane. The removal of the heptane under vacuum afforded 42.0 g of the bismaleimide resin, 60% yield. The FTIR spectrum of this compound was identical to that of a known pure sample of the bismaleimide of dimer diamine that had been prepared via a different cyclodehydration route.

### Example 2

### Preparation of Bismaleimide in the Presence of 100 mol % Alcohol Catalyst

The same procedure as set forth above in EXAMPLE 1 was followed except that 100 mol % (17.6 g) of n-pentanol was added. After workup a similar yield of 60% and a similar purity as in EXAMPLE 1 was obtained.

### Example 3

### Preparation of Benzylmaleimide

The general method of EXAMPLE 1 was followed except that 32.1 g of benzylamine was slowly added to a stirred solution of 31.0 g of maleic anhydride in 300-mL of toluene. After the maleamic acid had formed, 11.1 g (50-mol %) of n-butanol was added followed by the addition of 10.0 g of methanesulfonic acid. The solution was refluxed for 24 hours and approximately 7.0 mL of water and n-butanol was collected in the Dean-Stark trap. The workup consisted of washing the solution with a saturated NaHCO₃ solution followed by drying over MgSO₄. The solvent was removed under vacuum to afford the crude product, which solidified upon standing. The brown crystals were washed with cold methanol and dried, 49.2 g of benzylmaleimide was isolated (88% yield). The FTIR spectrum of the product was in agreement with known maleimide samples.

### Example 4

### Preparation of Polyimide

To a 1-L Erlenmeyer flask equipped with a stir bar was added 310 mmol Versamine-552 (dimer diamine). Pyrromelitic anhydride (300 mmol) was added to the flask along with toluene (400 g) and formic acid (50 mL). The mixture was stirred at room temperature for one hour to form the polyamic acid. The solution was then heated to reflux for 3 hours to complete the conversion to polyimide. In the Dean-Stark trap 61 mL of a second phase was recovered, which was equal to the theoretical amount of formic acid plus water expected from the reaction. The FTIR spectrum of the solution indicated the formation of polyimide.

### Example 5

### Preparation of Maleimde-Terminated Polyimide

To a 3-L 3-neck flask equipped with an electric stir motor, heating mantle and Dean-Stark trap with reflux condenser was added 643.2 g of dimer diamine (Versamine-552), which was dissolved in 1 Kg of toluene. Slowly 218.0 g of pyromellitic dianhydride was added to the solution and stirred vigorously, while 150.0 g of 95% formic acid was added slowly to the mixture. The mixture was heated to 50-60° C. for approximately one hour to allow all of the dianhydride to react and to allow the polyamic acid to form. To this solution, 49.0 g of maleic anhydride was added and stirred for an additional hour, followed by the addition of 30.0 g of dry Amberlyst-70 ion exchange resin and 14.8 g of n-butanol. The solution was slowly brought to reflux, and water, formic acid, n-butanol and toluene were collected in the Dean-Stark trap. After reflux for about 18 hours, no further water droplets were observed coming off of the reaction and the heat was shut off. The aqueous phase in the Dean-Stark trap was separated and determined to weigh approximately 200 g. The Amberlyst-70 resin was separated by filtration, and the maleimide-terminated polyimide was then isolated by precipitation in acetone. The FTIR spectrum of the product indicated the presence of polyimide and maleimide moieties.

### Example 6

### Preparation of Maleimde-Terminated Polyimide

To a 1-L round-bottomed flask, equipped with a Teflon coated stir bar, heating mantle and Dean-Stark trap with reflux condenser was added 52.0 g of bisphenol-A dianhydride along with 300.0 g of toluene and 50.0 g of 95% formic acid. To the mixture was slowly added 23.3 g of bis(aminomethyl)tricyclodecane, and stirred at 50° C. for one hour to complete the polyamic acid formation. At this point, 4.9 g of maleic anhydride was added and the solution was stirred for an additional hour to fully react the amino end groups. To the solution was then added 1.5 g (50 mol %) n-butanol, followed by 10 g of dry Amberlyst-70 ion exchange resin. The mixture was slowly heated to reflux and the water, formic acid, n-butanol and toluene were collected in the Dean-Stark trap. After 18 hours no more water was observed being generated in the reaction. The Amberylyst-70 resin was removed by gravity filtration, and the maleimide-terminated polyimide was isolated by precipitation in methanol. The FTIR spectrum of the product indicated the presence of polyimide and maleimide moieties.

### Example 7

### Preparation of Maleimide-Terminated Polyimide from a Mixture of Diamines

To a 1-L round bottomed flask equipped with a Teflon-coated stir bar, heating mantle and Dean-Stark trap with condenser was added 52.0 g of bisphenol-A dianhydride along with 300.0 g of toluene and 50 g of 95% formic acid. To the mixture was slowly added 43.2 g of Versamine-552 and the mixture stirred at 50° C. for 1 hour to complete the polyamic acid formation. At this point the solution was slowly heated to reflux and after 2 hours approximately 53 g of water and formic acid was collected in the Dean-Stark trap. The anhydride terminated polyimide was allowed to cool down just below reflux and 7.7 g of bis(aminomethyl)tricyclodecane was slowly added to the solution and stirred for 30 minutes to complete the reaction. The thick mixture was refluxed for an additional hour to complete the formation of the amine-terminated polyimide. The solution was cooled down to 50° C. and 4.1 g of maleic anhydride was added to the solution and stirred for 1 hour. This was followed by the addition of 1.5 g of n-butanol and 30 g of dry Amberlyst-70 catalyst. The mixture was refluxed again overnight for 14 hours to complete the ring closing to the maleimide with the collection of approximately 1 mL of water and n-butanol in the Dean-Stark trap. After cooling the reaction down, the Amberlyst-70 catalyst was filtered washed and recycled. The presence of the maleimide-terminated polyimide was confirmed by FTIR spectroscopy. The removal of the toluene under vacuum produced a thick leathery resin that thermally cured in the presence of free-radical initiators.

What is claimed is:

1. A method for preparing a maleimide, comprising:
a) contacting an N-substituted maleamic acid with an alcohol catalyst and an acid co-catalyst in a solvent, wherein the alcohol catalyst has a boiling point equal to or greater than the boiling point of the solvent; and
b) heating the reaction medium at an elevated temperature, wherein the heating results in cyclodehydration of the N-substituted maleamic acid, thereby preparing a maleimide.

2. The method of claim 1, wherein the alcohol catalyst is a C₄ to about C₁₀ alcohol.

3. The method of claim 1, wherein the alcohol catalyst is a C₄ alcohol.

4. The method of claim 1, wherein the alcohol catalyst is n-butanol.

5. The method of claim 1, wherein the alcohol catalyst is a C₅ alcohol.

6. The method of claim 1, wherein the alcohol catalyst is n-pentanol.

7. The method of claim 1, wherein the alcohol catalyst is present in the reaction medium in the range of 10 mol % to about 40 mol %, based on mol % of N-substituted maleamic acid.

8. The method of claim 1, wherein the acid co-catalyst is an organic sulfonic acid.

9. The method of claim 8, wherein the organic sulfonic acid is benzenesulfonic acid, para-toluenesulfonic acid, para-bromobenzenesulfonic acid, para-nitrobenzenesulfonic acid, ethylbenzenesulfonic acid, methanesulfonic acid, ethanesulfonic acid, propanesulfonic acid, trifluoromethanesulfonic acid, nonafluorobutanesulfonic acid, or 2,2,2-trifluoroethanesulfonic acid.

10. The method of claim 1, wherein the solvent has a boiling point of at least 100° C.

11. The method claim 1, wherein the solvent is an alkane, a cycloalkane, an aromatic hydrocarbon or halogen-substituted derivative thereof.

12. The method of claim 1 wherein the solvent is an aromatic hydrocarbon.

13. The method of claim 1, wherein the solvent is toluene.

14. A method for preparing a maleimide-terminated polyimide, comprising:
a) contacting at least one diamine with at least one anhydride in an aromatic solvent containing at least one polar protic co-solvent, wherein a polyamic acid is formed;
b) contacting the polyamic acid with maleic anhydride, thereby forming maleamic acid termini; and
c) contacting the maleamic acid termini with at least one acid catalyst, thereby forming a polyimide.

15. The method of claim 14, wherein the at least one acid catalyst is a polymer-supported acid catalyst.

16. The method of claim 14, further comprising the step of:
d) removing the polymer-supported acid catalyst by gravity filtration.

17. The method of claim 14, wherein the at least one diamine comprises: 1,10-diaminodecane; 1,12-diaminododecane; dimer diamine; 1,2-diamino-2-methylpropane; 1,2-diaminocyclohexane; 1,2-diaminopropane; 1,3-diaminopropane; 1,4-diaminobutane; 1,5-diaminopentane; 1,7-diaminoheptane; 1,8-diaminomenthane; 1,8-diaminooctane; 1,9-diaminononane; 3,3′-diamino-N-methyldipropylamine; diaminomaleonitrile; 1,3-diaminopentane; 9,10-diaminophenanthrene; 4,4′-diaminooctafluorobiphenyl; 3,5-diaminobenzoic acid; 3,7-diamino-2-methoxyfluorene; 4,4′-diaminobenzophenone; 3,4-diaminobenzophenone; 3,4-diaminotoluene; 2,6-diaminoanthroquinone; 2,6-diaminotoluene; 2,3-diaminotoluene; 1,8-diaminonaphthalene; 2,4-diaminotoluene; 2,5-diaminotoluene; 1,4-diaminoanthroquinone; 1,5-diaminoanthroquinone; 1,5-diaminonaphthalene; 1,2-diaminoanthroquinone; 2,4-cumenediamine; 1,3-bisaminomethylbenzene; 1,3-bisaminomethylcyclohexane; 2-chloro-1,4-diaminobenzene; 1,4-diamino-2,5-dichlorobenzne; 1,4-diamino-2,5-dimethylbenzene; 4,4′-diamino-2,2′-bistrifluoromethylbiphenyl; bis(amino-3-chlorophenyl)ethane; bis(4-amino-3,5-dimethylphenyl)methane; bis(4-amino-3,5-diethylphenyl)methane; bis(4-amino-3-ethyl diaminofluorene; diaminobenzoic acid; 2,3-diaminonaphthalene; 2,3-diaminophenol; -5-methylphenyl)methane; bis(4-amino-3-methylphenyl)methane; bis(4-amino-3-ethylphenyl)methane; 4,4′-diaminophenylsulfone; 3,3′-diaminophenylsulfone; 2,2-bis(4, -(4-aminophenoxy)phenyl)sulfone; 2,2-bis(4-(3-aminophenoxy)phenyl)sulfone; 4,4′-oxydianiline; 4,4′-diaminodiphenyl sulfide; 3,4′-oxydianiline; 2,2-bis(4-(4-aminophenoxy)phenyl)propane; 1,3-bis(4-aminophenoxy)benzene; 4,4′-bis(4-aminophenoxy)biphenyl; 4,4′-diamino-3,3′-dihydroxybiphenyl; 4,4′-diamino-3,3′-dimethylbiphenyl; 4,4′-diamino-3,3′-dimethoxybiphenyl; Bisaniline M; Bisaniline P; 9,9-bis(4-aminophenyl)fluorene; o-tolidine sulfone; methylene bis(anthranilic acid); 1,3-bis(4-aminophenoxy)-2,2-dimethylpropane; 1,3-bis(4-aminophenoxy)propane; 1,4-bis(4-aminophenoxy)butane; 1,5-bis(4-aminophenoxy)butane; 2,3,5,6-tetramethyl-1,4-phenylenediamine; 3,3′,5,5′-tetramethylbenzidine; 4,4′-diaminobenzanilide; 2,2-bis(4-aminophenyl)hexafluoropropane; polyoxyalkylenediamines (e.g. Huntsman's Jeffamine D-230, D400, D-2000, and D-4000 products); 1,3-cyclohexanebis(methylamine); m-xylylenediamine; p-xylylenediamine; bis(4-amino-3-methylcyclohexyl)methane; 1,2-bis(2-aminoethoxy)ethane; 3(4), 8(9)-bis(aminomethyl)tricyclo(5.2.1.0^{2,6})decane; or isophoronediamine.

18. The method of claim 14, wherein the at least one anhydride comprises: polybutadiene-graft-maleic anhydride; polyethylene-graft-maleic anhydride; polyethylene-alt-maleic anhydride; polymaleic anhydride-alt-1-octadecene; polypropylene-graft-maleic anhydride; poly(styrene-co-maleic anhydride); pyromellitic dianhydride; maleic anhydride, succinic anhydride; 1,2,3,4-cyclobutanetetracarboxylic dianhydride; 1,4,5,8-naphthalenetetracarboxylic dianhydride; 3,4,9,10-perylenentetracarboxylic dianhydride; bicyclo(2.2.2)oct-7-ene-2,3,5,6-tetracarboxylic dianhydride; diethylenetriaminepentaacetic dianhydride; ethylenediaminetetraacetic dianhydride; 3,3′,4,4′-benzophenone tetracarboxylic dianhydride; 3,3′,4,4′-biphenyl tetracarboxylic dianhydride; 4,4′-oxydiphthalix anhydride; 3,3′,4,4′-diphenylsulfone tetracarboxylic dianhydride; 2,2′-bis(3,4-dicarboxyphenyl)hexafluoropropane dianhydride; 4,4′-bisphenol A diphthalic anhydride; 5-(2,5-dioxytetrahydro)-3-methyl-3-cyclohexene-1,2-dicarboxylic anhydride; ethylene glycol bis(trimellitic anhydride); hydroquinone diphthalic anhydride; allyl nadic anhydride; 2-octen-1-ylsuccinic anhydride; phthalic anhydride; 1,2,3,6-tetrahydrophthalic anhydride; 3,4,5,6-tetrahydrophthalic anhydride; 1,8-naphthalic anhydride; glutaric anhydride; dodecenylsuccinic anhydride; hexadecenylsuccinic anhydride; hexahydrophthalic anhydride; methylhexahydrophthalic anhydride; or tetradecenylsuccinic anhydride.

19. The method of claim 14, wherein the aromatic solvent is toluene.

20. The method of claim 14, wherein the at least one polar protic co-solvent comprises formic acid.
